(19) United States
(12) Patent Application Publication
(10) Pub. No.: US 2012/0156312 A1
(43) Pub. Date: Jun. 21, 2012

(54) Compositions for Inhibiting Growth of Cancer Stem Cells

(75) Inventors: Michael Spinella, Hanover, NH (US); Maroun J. Beyrouthy, Lebanon, NH (US)

(73) Assignee: Trustees of Dartmouth College, Hanover, NH (US)

(21) Appl. No.: 13/393,290

(22) PCT Filed: Aug. 30, 2010
(86) PCT No.: PCT/US10/47140

§ 371 Date: Feb. 29, 2012

Related U.S. Application Data
(60) Provisional application No. 61/238,881, filed on Sep. 1, 2009.

(51) Int. Cl. A61K 31/706; A61P 35/00; A61K 33/24; C12N 9/10; C12N 5/095

(52) U.S. Cl. 424/649; 435/193; 435/375; 514/43

(57) ABSTRACT

The present invention is a biomarker of chemotherapeutic drug-resistant cancer stem cells and a method of inhibiting the growth of drug-resistant cancer stem cells. In one embodiment the cancer stem cells are testicular cancer germ cells. In another embodiment the present invention is a method of overcoming drug resistance in cancer treatment where the combination of low dose decitabine and administration of a chemotherapeutic drug to which cancer cells were resistant results in successful cancer treatment.

This application claims the benefit of priority to U.S. Provisional Application Ser. No. 61/238,881 filed Sep. 1, 2009, the contents of which are incorporated herein by reference. This invention was made in the course of research sponsored by the National Institutes of Health, grant number CA104312. The U.S. government has certain rights in this invention.

### BACKGROUND OF THE INVENTION

Recent evidence indicates that cells within a tumor are heterogeneous and represent different stages of development (Clarke et al. 2006. Cancer Res. 66:9339-9344). In certain types of cancer, a population of cells has been identified that are termed cancer stem cells, where a cancer stem cell is defined as a cell that has the capacity to self-renew and to cause the heterogeneous lineages of cancer cells that comprise a tumor. Experimentally, such cells are ones that have the ability to generate a continuously growing tumor (Clarke et al. 2006. Cancer Res. 66:9339-9344). Cancer stem cells can arise from normal stem cells but also from cells that acquire the capacity to self-renew potentially due to a series of mutagenic events within the cell. There is considerable interest in the role of cancer stem cells in certain types of cancer. Cancer types that have been associated with the presence of cancer stem cells include breast cancer (Al-Hajj et al. 2003. PNAS 100:3983-3988), pancreatic cancer (Hermann et al. 2007. Cell Stem Cell 1:313-323), brain cancer (Singh et al. 2004. Nature 432:396-401), and testicular cancer (Houldsworth et al. 2006. J. Clin. Oncol. 24:5512-5518; Clark A. T. 2007. Stem Cell Rev. 3:49-59.

Testicular germ cell tumors (TGCTs), the most common solid tumors of adolescent and young men, are thought to derive from transformation of primordial germ cells (PGCs) or early gonocytes (Houldsworth et al. 2006. J. Clin. Oncol. 24:5512-5518; Clark A. T. 2007. Stem Cell Rev. 3:49-59). TGCTs are classified as seminomas and nonseminomas (Houldsworth et al. 2006. J. Clin. Oncol. 24:5512-5518). Within nonseminomas are undifferentiated, pluripotent cells, known as embryonal carcinoma (EC) cells. EC cells are proposed to represent the stem cells of TGCTs and to be the malignant counterparts to embryonic stem (ES) cells (Houldsworth et al. 2006. J. Clin. Oncol. 24:5512-5518; Clark A. T. 2007. Stem Cell Rev. 3:49-59). EC cells can differentiate in vivo toward extra-embryonic tissues and embryonic tissues.

Patients with TGCTs, even those with advanced metastatic disease, are successfully treated with cisplatin-based chemotherapeutic regimens (Giuliano et al. 2006. Curr. Cancer Ther. Rev. 2:255-270; Einhorn, L. H. 2002. Proc. Natl. Acad. Sci. USA 99:4592-4595). However, 15-20% of patients are refractory to treatment and succumb to progressive disease (El-Helw, L. and R. E. Coleman. 2005. Cancer Treat. Rev. 31:197-209). Some germ cell tumor patients who initially respond to treatment can exhibit a late relapse and have a poor prognosis (Giuliano et al. 2006. Curr. Cancer Ther. Rev. 2:255-270; El-Helw, L. and R. E. Coleman. 2005. Cancer Treat. Rev. 31:197-209). Additionally, testicular cancer survivors have increased incidence of infertility, cardiovascular disease and secondary malignancies (Chaudhary et al. 2003. Drugs 63:1565-1577), all of which can affect ultimate survival and quality of life of testicular cancer patients. Mouse models of testicular cancer do exist, but they do no recapitulate key features of the human malignancy (Houldsworth et al. 2006. J. Clin. Oncol. 24:5512-5518).

Mechanisms of inherent or acquired cisplatin resistance in other tumors have not yet provided insights into the exquisite cisplatin-sensitivity of TGCTs (Giuliano et al. 2006. Curr. Cancer Ther. Rev. 2:255-270). That patients with advanced stage TGCTs can be cured implies that the stem cells of TGCTs are effectively targeted with cisplatin-based chemotherapy (Houldsworth et al. 2006. J. Clin. Oncol. 24:5512-5518; Giuliano et al. 2006. Curr. Cancer Ther. Rev. 2:255-270). There is a need to identify other chemotherapeutic agents for use in the patients that do not respond to cisplatin therapy, or that have become resistant to cisplatin therapy.

DNA methylation inhibitors, another class of chemotherapeutic agents, have been found to be more active in leukemia than in solid tumor cells (Qin et al. 2009. Blood 113:659-667). One such drug, 5-aza-deoxycytidine, also known as decitabine, has been shown to be useful for treating leukemia (e.g., Garcia-Manero, G. 2008. Curr. Opin. Oncol. 20:705-710). Decitabine is currently approved in the United States for the treatment of myelodysplastic syndromes which include leukemia. Although many papers describe the efficacy of decitabine in the treatment of leukemia, the published medical literature does not support the use of decitabine in the treatment of other types of cancer. For example, Abele et al. (1987. Eur. J. Cancer Clin. Oncol. 23:1921-1924) described the use of decitabine at a dose of 75 mg/m² (3 treatments in one day; repeated once a week for 5 weeks) for treatment of colorectal cancer, cancer of the head and neck, renal carcinoma, or malignant melanoma. The authors reported that decitabine showed no efficacy against any of the forms of cancer. In another study (Clavel et al. 1992. Ann Oncol. 3:399-400), decitabine was tested in a Phase II clinical trial in patients with non-seminiferous testicular cancer (i.e., germ cell testicular cancer). The authors used a decitabine dose of 75 mg/m² (3 infusions in one day, repeated once a week for 5 weeks; the standard leukemia regimen) and reported that the drug showed “no activity” in these patients.

U.S. Pat. No. 6,613,753 teaches administering the DNA methylation inhibitor decitabine, in combination with an anti-neoplastic agent, to treat cancer. A long list of cancers is disclosed, including testicular cancer. The patent teaches use of decitabine in combination with chemotherapeutic agents that include cisplatin and to treat cisplatin resistance. The patent teaches and claims a preferred dose range for decitabine of 1-20 mg/m²/day. No data are provided showing successful treatment of germ cell testicular cancer with this regimen.

### SUMMARY OF THE INVENTION

The present invention is a biomarker of decitabine-sensitive cancer stem cells comprising DNA methyltransferase 3B (DNMT3B). In a preferred embodiment the cancer stem cell is a testicular cancer germ cell, a breast cancer stem cell, a pancreatic cancer stem cell, or a glioblastoma stem cell.

Another object of the present invention is a method of inhibiting growth of chemotherapeutic drug-resistant cancer stem cells comprising contacting said cells with a low dose of decitabine and an effective dose of a chemotherapeutic drug to which said cancer stem cells are resistant. In a preferred embodiment the cancer stem cells are testicular cancer germ cells and the chemotherapeutic drug is cisplatin.

Yet another object of the present invention is a method for treating chemotherapeutic drug-resistant cancer comprising administering to a patient with chemotherapeutic drug-resistant cancer a low dose of decitabine in combination with a therapeutically effective dose of the chemotherapeutic drug to which said cancer is resistant so that the growth of cancer stem cells is inhibited. In a preferred embodiment the cancer is testicular germ cell cancer and the chemotherapeutic drug is cisplatin.

### DETAILED DESCRIPTION OF THE DRAWINGS

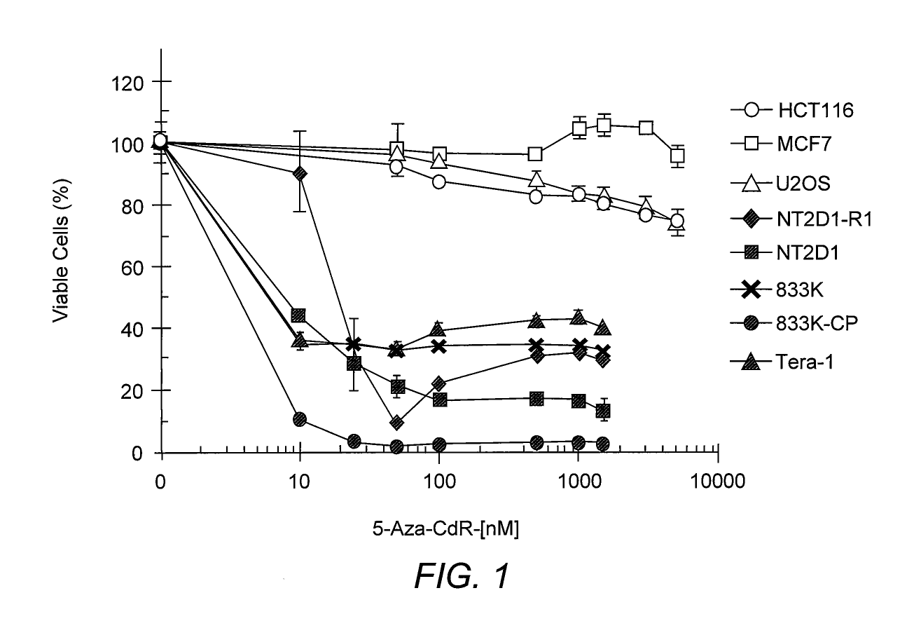

FIG. 1 depicts the results of experiments showing that EC cell lines are sensitive to low dose decitabine. Indicated doses of decitabine were added fresh each day for three days to exponentially growing cultures. Viable cell growth and survival were measured. Data are normalized to no drug treatment. EC cells are NT2/D1, NT2D1/R1, 833K, 833KCP, and Tera-1. Data are the average of experiments in biological duplicate except for MCF-7 cells, which were assayed twice. Error bars are S.D.

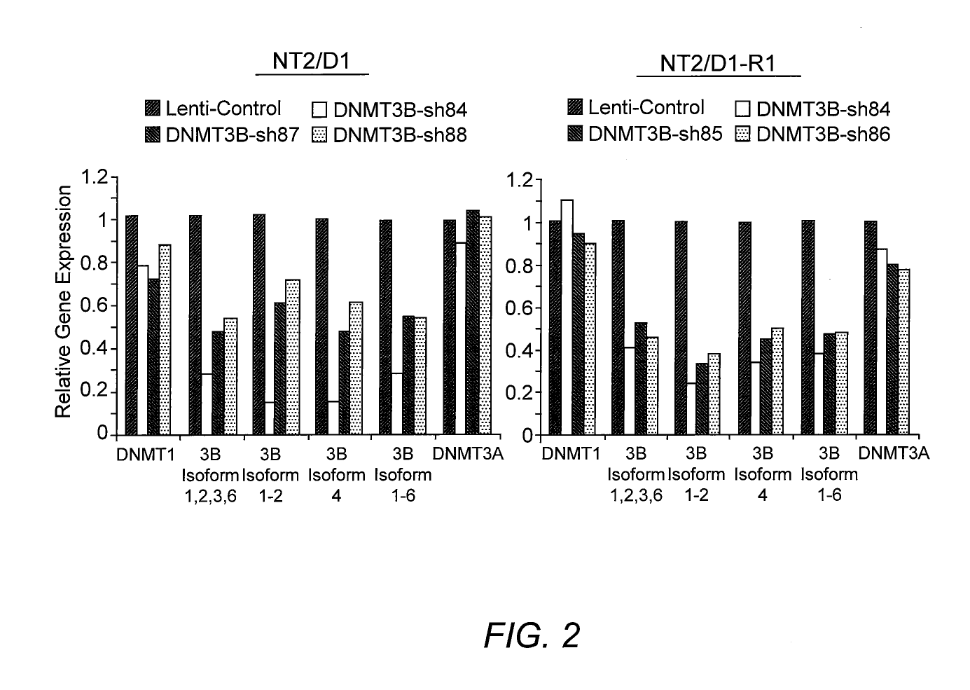

FIG. 2 depicts DNMT3B knock down results in resistance to decitabine in EC cells. Results of real-time PCR assays of DNMT3B isoforms in control NT2/D1 and in NT2/D1-R1 cells and cells treated with DNMT3B shRNA lentiviruses are shown. Knock down results in resistance to decitabine in EC cells.

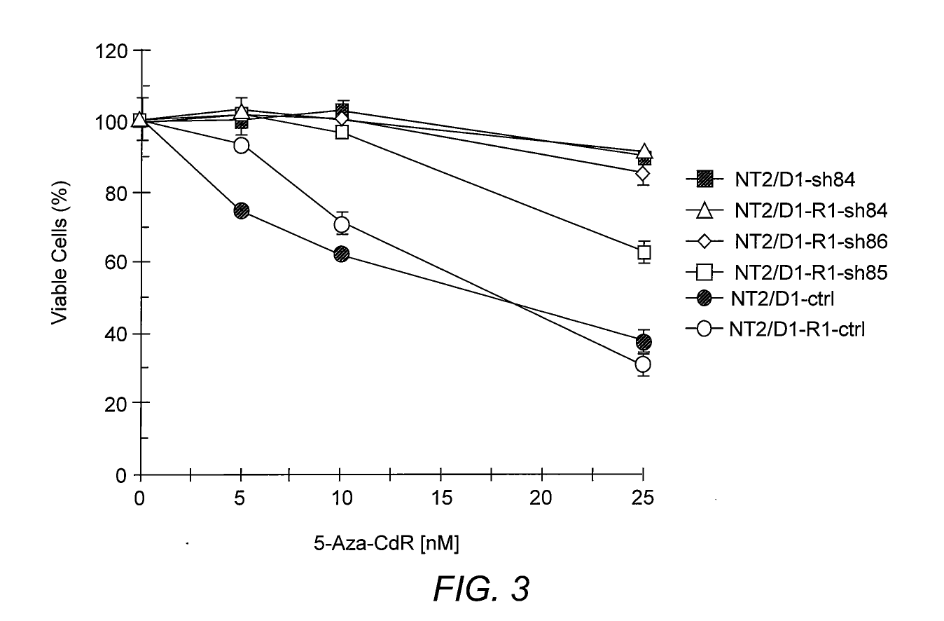

FIG. 3 depicts DNMT3B knock down results in resistance to decitabine in EC cells. Dose-response is observed after 3 day of decitabine treatment in lentiviral control NT2/D1 as well as NT2/D1-R1 cells (ctrl) and cells treated with DNMT3B sh84, 85 and 86. Data are the average of biological triplicates and are representative of at least two experiments. Error bars are S.D.

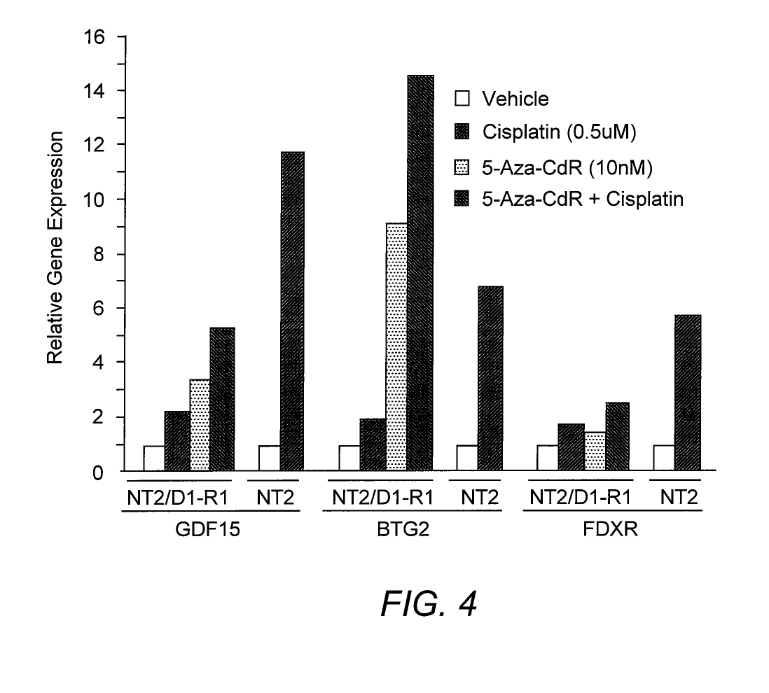

FIG. 4 depicts the effects of pretreatment with low dose decitabine to restore cisplatin sensitivity to cisplatin-resistant EC cells. NT2/D1-R1 cells were pretreated with vehicle or decitabine (10 nM) for 3 days before replating and 24 hour recovery followed by indicated cisplatin treatments for 6 hours. NT2/D1 cells were only treated with cisplatin. Cells were assayed 24 hours later for expression of indicated p53 target genes by real-time PCR assays.

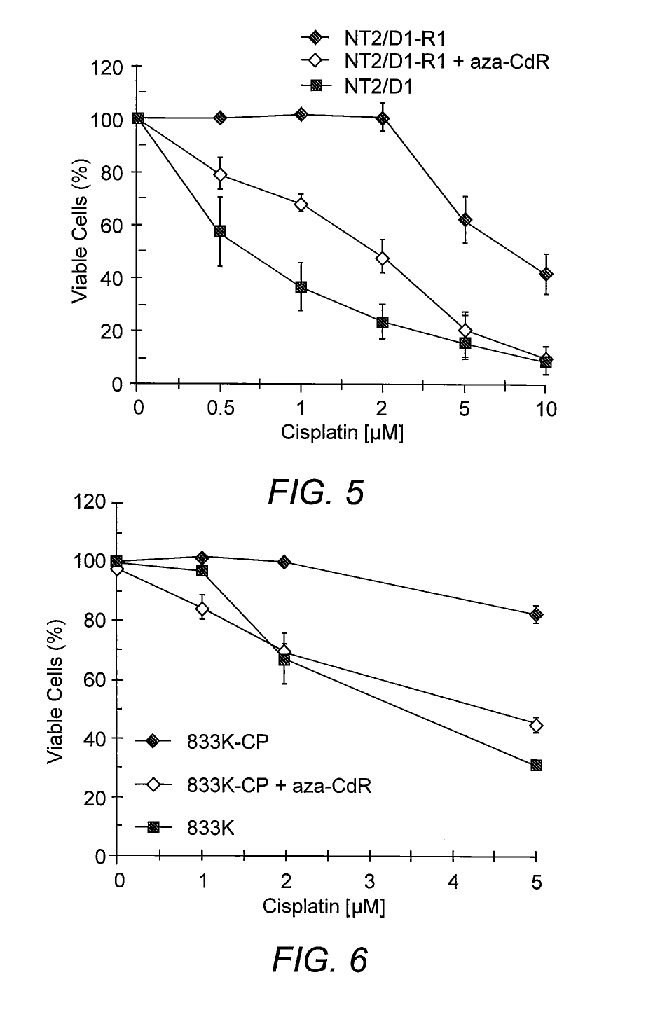

FIG. 5 depicts the effects of pretreatment with low dose decitabine to restore cisplatin sensitivity to cisplatin-resistant EC cells. Cells were pretreated with vehicle or decitabine for 3 days before replating and 24 hours recovery followed by indicated cisplatin treatments for 6 h. Cell viability was assayed 3 days later. For NT2D1-R1 cells, 10 nM decitabineR was employed. For 833K-CP cells, 2.5 nM decitabine was employed. Data are the average of biological triplicates and representative of at least two experiments. Error bars are SEM.

FIG. 6 also depicts the effects of pretreatment with low dose decitabine to restore cisplatin sensitivity to cisplatin-resistant EC cells. Cells were pretreated with vehicle or decitabine for 3 days before replating and 24 hours recovery followed by indicated cisplatin treatments for 6 h. Cell viability was assayed 3 days later. For NT2D1-R1 cells, 10 nM decitabineR was employed. For 833K-CP cells, 2.5 nM decitabine was employed. Data are the average of biological triplicates and representative of at least two experiments. Error bars are SEM.

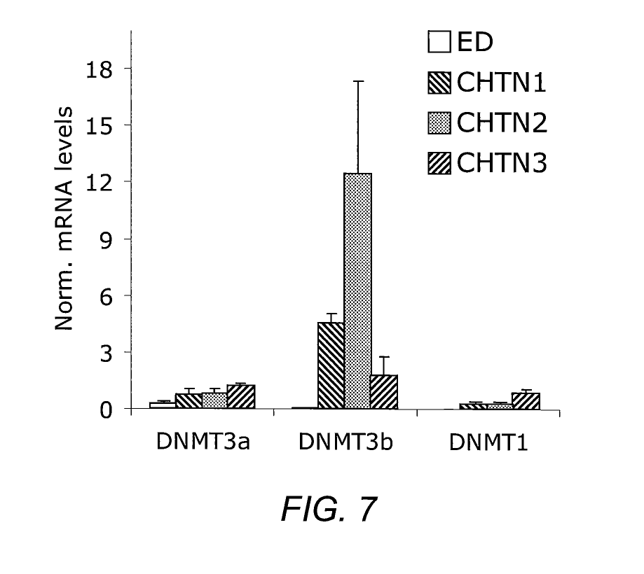

FIG. 7 depicts DNMT3B expression in clinical tumor samples. The graphs show levels of mRNA expression quantified with RT-PCR analysis of DNMTs. The samples tested included a mature teratoma (ED) and 3 different testicular germ cell tumors (denoted CHTN1 through CHTN-3, wherein CHTN is the Connective Human Tissue Network). Bars represent standard deviation from the mean of two determinations.

### DETAILED DESCRIPTION OF THE INVENTION

Recent evidence suggests that pluripotent embryonal carcinoma (EC) cells share many characteristics with rare cancer stem cells of common somatic cancers of the brain, breast and pancreas. These rare cancer stem cells are the cells that need to be targeted for cure. It has now been found that one type of cancer stem cells, TGCT cells, are extremely responsive to the DNA methylation inhibitor decitabine (5-aza-deoxycytidine or 5-aza-CdR). Doses of decitabine that are at least an order of magnitude lower than doses used clinically to treat leukemia (e.g., doses in the low nanomolar range) have been found to be effective in inhibiting growth of TGCT cells. The hypersensitivity of TGCT cells was also found to be associated with high levels of expression of the pluripotency-associated DNA methyltransferase 3B (DNMT3B). Thus increased expression of DNMT3B is a biomarker of sensitivity to low dose decitibine in the cancer stem cells of testicular cancer (i.e. EC cells). Moreover, the same sensitivity is expected for cancer stem cells of other tumor types.

Therefore, the present invention is a marker for cancer stem cell tumor cells that can be successfully treated with decitabine in combination with a chemotherapeutic drug, and result in re-sensitization of chemotherapeutic drug-resistant tumor cells to drug-mediated cytotoxicity (i.e., chemotherapeutic drug efficacy in cancer treatment). In a preferred embodiment the cancer stem cells are testicular tumor cells and the chemotherapeutic drug is cisplatin. As a result, the present invention is a method of inhibiting the growth of chemotherapeutic drug-resistant cancer stem cells comprising contacting the cancer stem cells with a low dose of decitabine and an effective amount of a chemotherapeutic drug to which said cancer stem cells are resistant. Again, in a preferred embodiment the cancer stem cells are testicular cancer cells and the chemotherapeutic drug is cisplatin. The present invention is also a method of treatment of cancer in patients that have developed a resistance to chemotherapy, wherein a low dose of decitabine is administered in combination with the chemotherapeutic drug to which the cancer has become resistant. In a preferred embodiment the cancer is testicular germ cell cancer and the chemotherapeutic drug is cisplatin.

In the context of the present invention, a “low dose” of decitabine is defined as a dose that is at least an order of magnitude lower than the doses that have been used for treatment of leukemia (10 to 20 mg/m²/day; current labeling for the product in the Physician's Desk Reference and as discussed in Kantarjian et al. 2007. Blood 109:52-57). In the context of the present invention, an “effective amount” and a “therapeutically effective” dose of a chemotherapeutic drug, such as cisplatin, are defined as being doses that are used routinely by physicians in the treatment of cancer (i.e., for testicular cancer 20 mg/m²/day×5 days is used; Kondagunta, G. V. et al. 2005. J. Clin. Oncol. 23:9290-9294). Other therapeutically effective doses of other chemotherapeutic drugs that are typically used in cancer can be found in sources such as the Physician's Desk Reference, a reference that lists commonly used doses of drugs approved for use by the U.S. Food and Drug Administration as part of the drug labeling included in the reference. Further, one of skill in the art would be familiar with choosing such dosing regimens based upon their own experience with patients. Also in the context of the present invention, “DNMT3B” is defined as being any isoform of the protein that has been identified. Cisplatin use in the present invention represents conventional cytotoxic therapy that produces its anti-cancer effects by generating DNA damage or other genotoxic stress within cancer cells. However, one of skill in the art would understand that the findings described in the present invention with cisplatin would likely apply to other cytotoxic drugs. For example, testicular cancer patients are treated with a cocktail of drugs that include cisplatin, etoposide, vinblastine and bleomycin. Patients that become resistant to cisplatin usually also become simultaneously resistant to these other cytotoxic drugs as well. Thus, cisplatin-resistant cancer stem cells explored in the experiments described with the present invention would also be resistant to the other commonly used cytotoxic drugs. Therefore, the ability of low dose decitibine to reverse cisplatin resistance extends to reversing resistance to other cytotoxic drugs and even the entire class of cytotoxic drugs, which includes but is not limited to cisplatin, etoposide, vinblastine and bleomycin.

Experiments were first performed to determine whether various EC cell lines were sensitive to DNA methylation inhibition using decitabine. Cell growth and viability of five EC cell lines (NT2/D1, NT2D1/R1, 833K, 833KCP, and Tera-1) were determined, including two cell lines that are cisplatin-resistant (NT2/D1-R1 and 833KCP). Three control somatic tumor cell lines were also tested (HCT116, MCF7 and U20S). Doses of decitabine from 10 to 5000 nM were added fresh each day for three days to exponentially growing cells in culture. Viable cell growth and survival were measured using Cell-Titre Glo (Promega) assays. As shown in FIG. 1, growth of all EC cell lines was inhibited with decitabine treatment. The NT2/D1 and 833K cell lines were the most highly sensitive to decitabine treatment, with IC₅₀ values calculated to be in the range of 5 to 25 nM (FIG. 1). These doses are substantially lower that the doses routinely reported for growth inhibition of solid somatic tumors exposed to decitabine which are typically in the range of 500 nM to 10 μM (Qin et al. 2009. Blood 113:659-667; Shen et al. 2007. Cancer Res. 67:11335-11343). These higher values are similar to the values shown in FIG. 1 for somatic tumor cell lines (MCF7, U2OS and HCT116). Interestingly, the cell line most sensitive to decitabine treatment was the cisplatin-resistant cell line, 833K-CP (also known as 833K64-CP10).

Recent microarray studies have indicated that ES cells and EC cells, as well as clinical EC cells and non-seminomas, express high levels of mRNA for DNMT3B as compared to expression levels seen in normal and somatic tumors (Sperger et al. 2003. Proc. Natl. Acad. Sci USA 100:13350-13355; Muller et al. 2008. Nature 455:401-405; Skotheim et al. 2005. Cancer Res. 65:5588-5598). However, this differential expression has not been confirmed or shown at the protein level. Therefore, experiments were performed to determine the level of DNMT3B protein expression in the various EC cell lines as compared to the somatic tumor cell lines previously tested. Western blot analysis was used to determine expression levels of DNMT3B in the EC cell lines (NT2/D1, NT2/D1-R1, 833K, 833KCP, Tera-1, and 2102EP) as well as in the somatic cell lines HCT116, U2OS and MCF7, and the lung cancer cell lines HOP62, H197, U1752, A549 and H157. The DNMT3B antibodies ab2851 and H-230 were used in the analyses. A striking difference in DNMT3B protein expression was found in the EC cell lines NT2/D1, NT2/D1-R1, 833K, 833K-CP, Tera-1 and 2102EP as compared to the somatic tumor cell lines HCT116, U20S, MCF7 and the six lung cancer cell lines tested. Importantly, the high expression of DNMT3B in EC cells could be repressed with a shRNA specific for DNMT3B and could be detected with two distinct DNMT3B antibodies. Densitometry measurements revealed at least a 30-fold increase in DNMT3B expression in the EC cells as compared to somatic tumor cells. Thus, the hypersensitivity of TGCTs to low dose decitabine was shown to be associated with high expression of DNMT3B in EC cells. These data indicated that expression of high levels of DNMT3B in tumor cells is a biomarker for cells that are especially sensitive to decitabine growth inhibition

In order to confirm the connection between decitabine growth inhibition sensitivity and high levels of DNMT3B expression in EC cells, experiments were performed where DNMT3B expression was knocked down. Five distinct lentiviral shRNAs for DNMT3B (sh84, sh85, sh86, sh87, and sh88) were used to knock down DNMT3B expression. Six potential alternatively spliced isoforms of DNMT3B exist; the most biologically relevant isoforms are variants 1, 2, 3 and 6 (Jones, P. A. and S. B. Baylin. 2007. Cell 128:683-692). Quantitative RT-PCR assays employing isoform-specific primers revealed that the shRNAs (relative to controls) reduced expression of the DNMT3B isoforms. Western blot analysis of NT2/D1 and NT2/D1-R1 cells was also performed with cells treated with DNMT3B shRNA (sh84, sh85, sh86, sh88, sh84, sh87, sh88) and employing DNMT3B antibody, H-230. The results confirmed the reduced expression of DNMT3B when the shRNA were employed. None of the DNMT3B-specific shRNAs affected levels of DNMT1 or DNMT3A (FIG. 2). DNMT3B targeting shRNAs also reduced DNMT3B protein in both NT2/D1 and NT2/D1-R1 cells. Since NT2/D1 cells stably expressing sh84 and NT2/D1-R1 cells stably expressing sh84, sh85 and sh86 had the most efficient knock down of DNMT3B expression (FIG. 2), these cells were tested for decitabine sensitivity. It was found that cells expressing DNMT3B-targeting shRNAs exhibited dramatic reduction of decitabine sensitivity as compared to control cells (FIG. 3). However, knockdown of DNMT3B by itself had no apparent effect on the growth of NT2/D1 or NT2/D1-R1 cells. These results strongly support a functional link between sensitivity of EC cells to decitabine and high DNMT3B expression in these same cells. These data also indicated that expression of high levels of DNMT3B in tumor cells is a biomarker for cells that are especially sensitive to decitabine growth inhibition.

With the link between decitabine sensitivity and DNMT3B expression established, experiments were then performed to examine the effects of decitabine in restoring cisplatin sensitivity to cisplatin-resistant tumor cells using decitabine. It had been previously reported that cisplatin causes a global p53-dominant transcriptional response in EC cells (Kerley-Hamilton et al. 2005. Oncogene 24:6090-6100). Through microarray and other studies it was found that the p53 response is repressed in NT2/D1-R1 cells despite having abundant wild-type p53 expression (Curtin et al. 2001. Oncogene 20:2559-2569; Kerley-Hamilton et al. Biochim. Biophys. Acta 2007. 1769:209-219; Kerley-Hamilton et al. 2005. Oncogene 24:6090-6100). With these new experiments, it was shown that pre-treatment of NT2/D1-R1 cells with low dose (10 nM) decitabine for 3 days at least partially restored cisplatin (treatment for 6 hours) induction of the p53 target genes GDF15, BTG2 and FDXR in NT2/D1-R1 cells (FIG. 4). This dose of decitabine had been shown to inhibit proliferation of NT2/D1-R1 cells by only 10% versus control (FIG. 1). Viable cells were counted and replated after decitabine treatment and allowed to recover for 24 hours before cisplatin treatment. Results showed that pretreatment with low dose decitabine restored cisplatin-induced growth suppression and toxicity to two separate cisplatin-resistant cell lines, NT2/D1-R1 and 833K-CP (FIGS. 5 and 6). 833K-CP cells were pretreated with 2.5 nM 5-aza-CdR, a dose that results in a 10% growth inhibition (FIG. 1). These data demonstrated that decitabine treatment of cisplatin-resistant tumor cells restores cisplatin sensitivity as measured by a cytotoxic response in cisplatin-resistant EC cells. Therefore, contacting cisplatin-resistant EC cells with low doses of decitabine before cisplatin is a method of inhibiting tumor cell growth in the drug-resistant cells.

With data demonstrating that DNMT3B was a tumor marker in established in vitro tumor cell lines, the overexpression of DNMT3B in tumor cell lines was then confirmed in clinical samples (FIG. 7). Using quantitative RT-PCR methods, mRNA levels of DNMT3A, DNMT3B and DNMT1 were quantified in a mature teratoma sample (ED) and three different testicular germ cell tumors (denoted CHTN1 through CHTN-3, wherein CHTN is the Connective Human Tissue Network). As can be seen in FIG. 7, DNMT3B expression was increased in the testicular germ cell tumor clinical samples, indicating that this protein is a marker for tumors in vivo as well as in vitro.

Therefore, these experiments have shown that TGCT cells are hypersensitive to the DNA methylation inhibitor decitabine. Further, it has been shown that this response was integrally associated with very high levels of DNMT3B protein, validating this protein as an important target of decitabine-mediated hypersensitivity in cisplatin-sensitive as well as cisplatin-resistant TGCT cells. These data indicate that TGCT cells may be distinctly sensitive to DNA methylation inhibitors due to high levels of DNMT3B that are likely a result of the primary germ cell origins of EC cells and their similarities to ES cells, which also are known to express high levels of DNMT3B (Sperger et al. 2003. Proc. Natl. Acad. Sci. USA 100:13350-13355; Muller et al. 2008. Nature 455:401-405). This finding is consistent with the fact that genomic studies have highlighted DNMT3B as a marker of pluripotency (Sperger et al. 2003. Proc. Natl. Acad. Sci. USA 100:13350-13355; Muller et al. 2008. Nature 455:401-405).

The present invention is not limited, however, as a biomarker for TGCT cells. It has been shown that DNMT3B is classified as a cancer stem cell marker (Adewumi et al. 2007. Nat. Biotechnol. 25:803-816). Thus, it is likely that DNMT3B will prove to be a biomarker of cancer stem cells of other cancers as well. Thus, it is contemplated that one of skill would understand that the present invention includes biomarkers for cancer stem cells which would include but not be limited to TGCTs, breast cancer stem cells, pancreatic cancer stem cells, and brain cancer stem cells.

Therefore, also contemplated by the present invention is a method for treatment of cancer that has become resistant to chemotherapy. In a preferred embodiment the cancer is testicular cancer in men whose tumors have been shown to be cisplatin-resistant. One of skill would understand how to treat cancer with decitabine since decitabine is currently approved for the treatment of myelodysplastic syndrome and shows promise for the treatment of certain forms of leukemia. Recent data suggests that decitabine is most efficacious against leukemia when given chronically and at lower doses (less than 20 mg/m²/day) (Issa, J. P. 2007. Clin. Cancer Res. 13:1634-2637). However, in the present invention, it is contemplated that the doses of decitabine used will be at least an order of magnitude lower than those currently used for treatment of cancer. Further, it has now been found that high levels of DNMT3B expression in EC cells is a consequence of their pluripotent and germ cell origin that results in hypersensitivity to DNA methylation inhibitors. The finding that cisplatin-resistant EC cells retain a high degree of sensitivity to low dose decitabine and that pretreatment of decitabine restores cisplatin sensitivity (cytotoxicity) in cisplatin-resistant EC cells is an important advance. It is also contemplated that decitibine would be effective in treating resistance to other drugs in cancers associated with cancer stem cells, such as testicular cancer, since it has been shown that germ cell tumors are very often resistant to more than one drug (Giuliano et al. 2006. Curr. Cancer Ther. Rev. 2:255-270. As a result, the present invention includes methods of treating testicular cancer in men whose tumors are resistant to other drugs that would include but not be limited to etoposide, vinblastine and bleomycin. Finally, it is contemplated that TGCTs, as well as other cancer stem cells, would be sensitive to other epigenetic inhibitors, apart from decitibine, compounds that would include but not be limited to zebularine, 5-aza-CR, and other 5-aza (decitibine) analogues.

It is contemplated that one of skill in the art will demonstrate the anti-neoplastic efficacy of decitabine, or other related epigenetic inhibitors, in the most appropriate in vivo model system depending on the type of drug product being developed. For example, some in vivo models are more amenable to intravenous injection, the method of use for the drugs of the present invention. The medical literature provides detailed disclosure on the advantages and uses of a wide variety of such models.

Once the combination of drugs of the present invention has been shown to be effective in vivo in animals, clinical studies can be designed based on the doses shown to be safe and effective in animals. One of skill in the art will design such clinical studies using standard protocols as described in textbooks such as Spilker (2000. Guide to Clinical Trials. Lippincott Williams & Wilkins: Philadelphia).

It is also contemplated that one of skill in the art would explore the activity of decitabine in other cancers linked with cancer stem cells (e.g., brain, breast or pancreatic cancer). Thus, experiments are being performed in other cancer stem cells to show activity of decitabine. Experiments will be performed in CD133+ cancer stem cells from human glioblastoma cell lines. The experiments will determine expression levels of DNMT3B in these highly tumorigenic cells, as well as the sensitivity of these cells to low dose decitabine treatment. Experiments will also demonstrate the activity of decitabine to increase the sensitivity of the cancer stem cells to conventional cytotoxic chemotherapeutic agents, such as cisplatin. Such experiments will demonstrate that low dose decitabine therapy will be a non-toxic method for targeting cancer stem cells in various types of cancer.

The following non-limiting examples are provided to further illustrate the present invention.

### EXAMPLES

### Example 1

### Cell Culture and Drug Treatments

NT2/D1, NT2D1-R1, 833K, 833K-CP, Tera-1, U20S, and HCT116 cells were cultured in DMEM with 10% FBS supplemented with glutamine and antibiotics except for MCF7 cells that were cultured in F12-DMEM. The derivation of the NT2/D1-resistant NT2/D1-R1 cell line has been previously described (Curtin et al. 2001. Oncogene 20:2559-2569; Kerley-Hamilton et al. 2007. Biochim. Biophys. Acta 1769:209-219). Cells were treated with the indicated dosages of 5-azadeoxycytidine (5-aza-CdR) for 3 days. This drug was replenished each day. Cisplatin (Bristol Laboratories) treatments were performed at the concentrations and time points indicated. To assess cell proliferation and survival, Cell-Titre Glo (Promega) assays were performed.

### Example 2

### Real-time PCR and Western Blot Analysis

Reverse transcription (RT) was performed on 1 μg RNA using the Taqman RT kit (Applied Biosystems). Twenty ng of the resulting cDNA was used with SYBR green (Applied Biosystems) for quantitative real-time PCR assays utilizing the ddCT method normalized to GAPDH and the ABI Prism Sequence Detection System 7700. For Western analysis, cells were lysed in a radioimmune precipitation buffer, separated by SDS-PAGE, as previously described (8, 9). Antibodies to DNMT3B (H-230; sc-20704, Santa Cruz, and Ab2851, Abcam) and actin (C-1; sc01615, Santa Cruz) were employed.

### Example 3

### Lentiviral Production

Silencing shRNAs to human DNMT3B were purchased (Open Biosystems). Lentiviral particles were generated as previously described and cells were selected in 1.0 μg/ml puromycin (Sigma Chemical Company, St. Louis, Mo.) (Kerley-Hamilton et al. 2007. Biochim. Biophys. Acta 1769:209-219).

What is claimed is:

1. A biomarker of decitabine-sensitive cancer stem cells comprising DNA methyltransferase 3B (DNMT3B).

2. The biomarker of claim 1 wherein said cancer stem cell is a testicular cancer germ cell, a breast cancer stem cell, a pancreatic cancer stem cell, or a glioblastoma stem cell.

3. A method of inhibiting growth of chemotherapeutic-resistant cancer stem cells comprising contacting said cells with a low dose of decitabine and an effective dose of a chemotherapeutic drug to which said cancer stem cells are resistant.

4. The method of claim 3 wherein said cancer stem cells are testicular germ cell cancer cells and the chemotherapeutic drug is cisplatin.

5. A method for treating chemotherapeutic drug-resistant cancer comprising administering to a patient with chemotherapeutic drug-resistant cancer a low dose of decitabine before administration of a therapeutically effective dose of the chemotherapeutic drug to which said cancer is resistant so that the growth of cancer stem cells is inhibited.

6. The method of claim 5 wherein said cancer stem cells are testicular cancer germ cells and said chemotherapeutic drug is cisplatin.
